(19) United States
(12) Patent Application Publication
(10) Pub. No.: US 2006/0100581 A1
(43) Pub. Date: May 11, 2006

(54) Reservoir for front end loaded infusion device

(76) Inventor: Lasse Wesseltoft Mogensen, Soborg (DK)

Correspondence Address: BRINKS HOFER GILSON & LIONE, P.O. BOX 10395, CHICAGO, IL, 60610, US

(21) Appl. No.: 10/920,050

(22) Filed: Aug. 17, 2004

(51) Int. Cl. A61M 37/00

(52) U.S. Cl. 604/154

(57) ABSTRACT

A medical reservoir for a front loaded infusion device including a housing, a chamber inside the housing for receiving the reservoir and a drive member. The reservoir includes a first end; an outlet disposed at the first end, the outlet having closable dispensing means; a second open end; an elongate body portion disposed between the first and second ends; and a piston slideably arranged within the elongate body portion and being connectable to the drive member.

### FIELD OF THE INVENTION

The invention relates to the field of medical reservoirs of the type comprising a first end, an outlet disposed at said first end, a second open end, an elongate body portion disposed between said first and second ends, and a piston slideable arranged within said elongate body portion. More particularly, the invention relates to a medical reservoir adapted for an infusion device, adapted for front end loaded medical reservoirs.

### BACKGROUND

Infusion devices and systems are well known in the medical arts, for use in delivering or dispensing a prescribed medication such as insulin to a patient. Typically such devices comprise a pump housing adapted to receive a replaceable syringe or reservoir containing a prescribed medication for administration to a patient through infusion tubing and an associated catheter or infusion set.

Pump housings typically includes a drive motor connected for driving a reservoir piston to administer the medication to the patient. Programmable controls are normally provided for operating the drive motor continuously or at periodic intervals to obtain a closely controlled and accurate delivery of the medication over an extended period of time.

In the field of infusion devices the need for ever smaller devices is imminent. Small, compact devices are desirable because they appear less intimidating to the patient, they are more easily handled than large apparatuses and they can be carried on a patient, providing the patient with more freedom of movement.

Most infusion devices are configured such that the entire housing or parts thereof can be opened to provide access to a chamber for receiving the reservoir, such that the reservoir is loaded sideways into the infusion device.

An example of such a device is described in U.S. Pat. No. 5,505,709. In such a device, however, ample space must be reserved for means for holding the reservoir, and for means for opening the device, e.g. hinges.

Infusion devices adapted for front loading the reservoir can be made more compact than side loaded devices, because the above mentioned features can be omitted. Front loaded infusion devices and medical reservoirs [therefore] are known from U.S. Pat. No. 5,947,935. Such a device has a reservoir located outside the infusion device as an extension thereof, like the barrel of a gun. Such reservoirs have means disposed at their rear end for connecting the reservoir to an infusion device. This leaves the reservoir exposed to unintentional blows, which is disadvantageous, since it might damage the reservoir or the device.

U.S. Pat. No. 4,267,836 teaches a front loaded infusion device in which a reservoir during use is enclosed within the housing of the device. The replaceable ampoule has a piercable septum in a first end a slideable piston closing the second end. The infusion device comprise a housing including a chamber for receiving the ampoule, a drive member for moving the piston of the ampoule, and powered means for driving the drive member. The ampoule is loaded from the front end of the device, and a cap having piercing means for piercing the septum of the ampoule, infusion tube connector means for connecting an infusion tube, and means for securing the cap to the reservoir is utilized to secure the ampoule to the housing of the infusion device.

The cap taught in this document, however is disadvantages in that it takes up a lot of space on the infusion device as such. Further the effective length of the reservoir is unintentionally shortened, due to the space that the cap take up. Further, this device has a rather complex mechanism (the cap) for mounting the reservoir, comprising multiple, expensive components. When mounting the reservoir in the apparatus, the can must be kept separate from the apparatus itself, with the risk that the cap parts is misplaced or dropped, while handling the reservoir into the pump housing. If dropped e.g. on the floor the cap may be contaminated.

Another front loaded infusion device is known from U.S. Pat. No. 6,248,093.

Thus there is a need for a reservoir configured in such a way that the combination of the infusion device and the reservoir can be made more compact, and from fewer, more cost efficient parts. There is also a need for a reservoir configured such that the infusion device outer appearance will have as few protrusions as possible. Further, there is a need for a reservoir configured such that the effective volume of the reservoir can be maximized within the front loaded infusion device.

### BRIEF SUMMARY OF THE INVENTION

The object of the present invention is to overcome the aforementioned problems of the prior art. It is a further object of the invention to provide an alternative to existing medical reservoirs.

The object of the invention is achieved by a medical reservoir for a front loaded infusion device comprising a housing, a chamber inside said housing for receiving said reservoir and a drive member, said reservoir comprising a first end; an outlet disposed at said first end; a second open end; an elongate body portion disposed between said first and second ends; and a piston slideable arranged within said elongate body portion and being connectable to said drive member, where said reservoir further comprises sealing means disposed at the first end of said reservoir for sealing/closing said chamber, connecting means for securing the reservoir in the housing, and closable dispensing means. Thereby a very compact closure of the chamber is achieved, further allowing a large effective volume of the reservoir. The reservoir according to the invention thus forms its own closure of a reservoir chamber of an infusion device/pump. Such a reservoir further is much easier to operate by the patient or the medical staff because of the fewer parts when a cap or lid is eliminated.

### BRIEF DESCRIPTION OF THE DRAWINGS

The invention will be described in detail in the following with reference to the drawings.

FIG. 1 is an exploded perspective view of an exemplary embodiment of a medical reservoir and a piston rod;

FIG. 2 is a sectional exploded perspective view of the exemplary embodiment of a medical reservoir and a piston rod shown in FIG. 1;

FIG. 3 is a sectional exploded side view of the exemplary embodiment of a medical reservoir and a piston rod shown in FIGS. 1 and 2; and

FIG. 4 is a schematic outline of an infusion device containing a reservoir according to the invention.

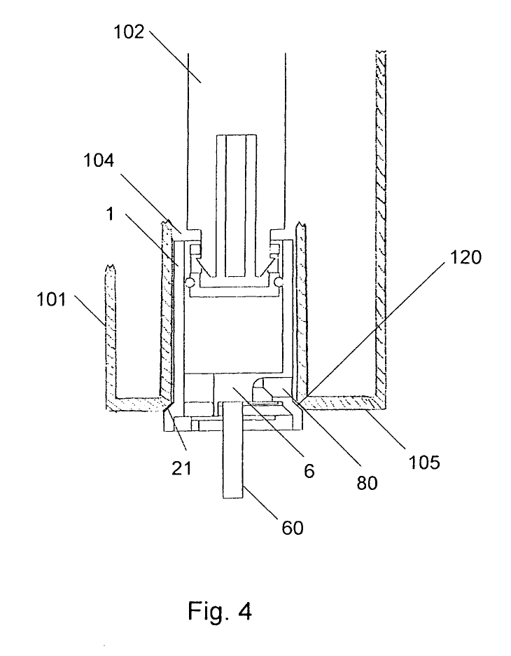

The drawings illustrate an embodiment of the present invention. It is understood that other embodiments may be utilized and structural and operational changes may be made without departing from the scope of the present invention.

### DETAILED DESCRIPTION OF THE INVENTION

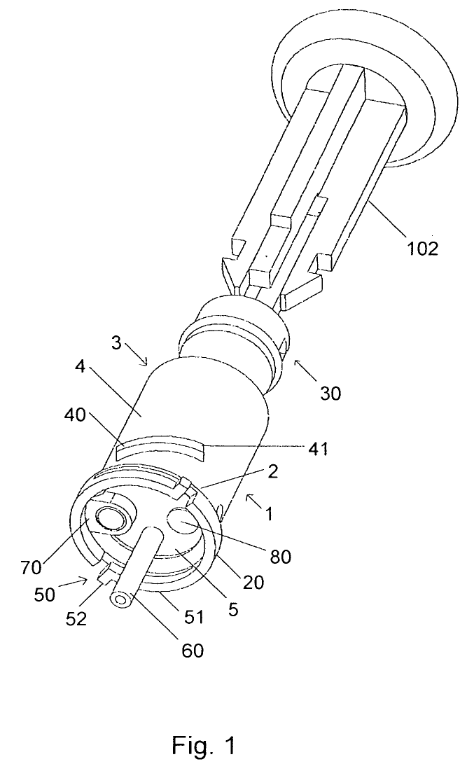

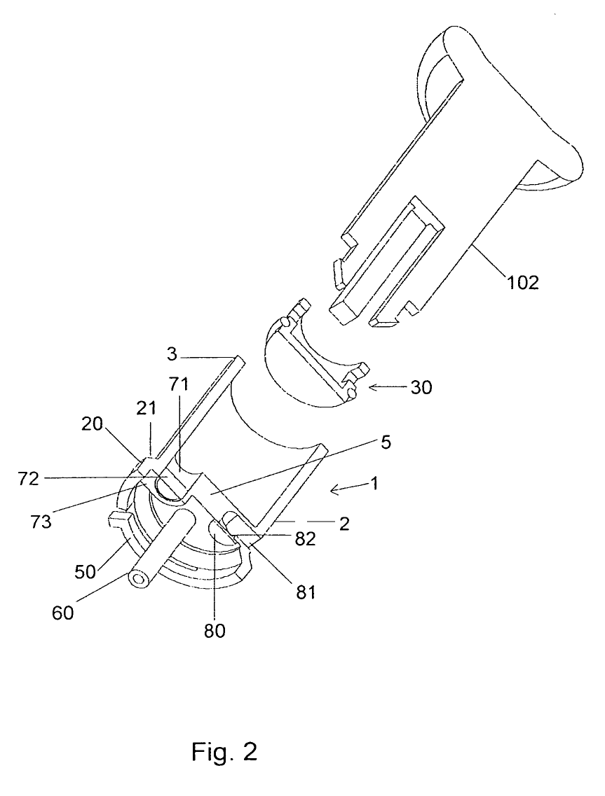

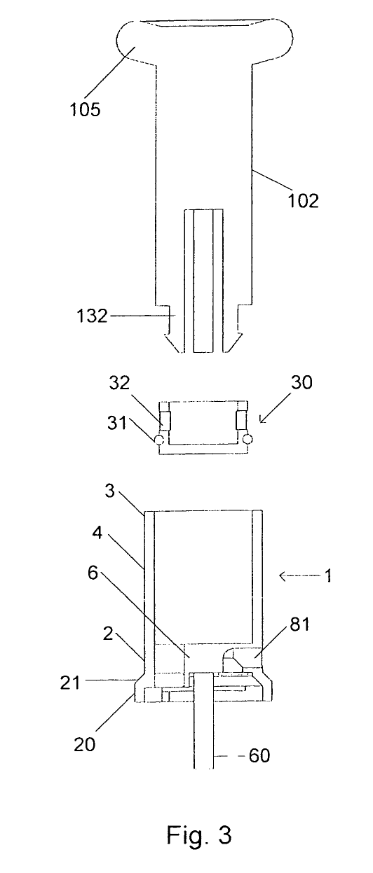

Referring firstly to FIGS. 1-3, a medical reservoir 1 according to an exemplary embodiment of the invention and a piston rod or drive member 102 is shown. The medical reservoir 1 comprises an elongate body portion 4, having first 2 and second 3 ends, and a piston 30 slideably arranged within said body portion 4. The second end 3 of the body portion 4 is open. The first end is closed e.g. by a wall 5. However, an outlet 6 is disposed therein. The piston 30 comprises sealing means 31 for providing a sealing between the piston 30 and the inner wall of the body portion 4. The body portion 4, the front wall 5 and the piston 30 thus forms a variable volume medical reservoir 1 for containing and dispensing a medical fluid such as e.g. insulin.

The piston 30 may further comprise means 32 for releasably connecting the piston 30 to a drive member 102. The drive member 102 is preferably a part of a medical injection or infusion device 100, having connection means 132 complementary to those on the piston 30. In FIGS. 1-3, however, the piston rod 102 is shown comprising a handle knob 105 for manual handling. Such a piston rod 102 with a handle knob 105 can be used to test the reservoir 1. The piston rod 102 of the injection or infusion device for witch the reservoir is intended of course does not necessarily comprise such a knob 105 but has means for cooperating with a drive mechanism of the infusion device 100.

The outlet 6 disposed in front wall 5 of the reservoir 1 communicates with and is connected to closable dispensing means. In an embodiment of the present invention said dispensing means consist of a length of infusion tubing 60. Said infusion tubing 60, may be fixedly mounted to the front end 2 of the reservoir 1, preferably in the front wall 5, and in fluid communication with outlet 6. Such a reservoir, where the infusion tubing 60 is integrated with the front end of the reservoir 1 is advantageous in that it can be delivered as an integrated aseptic set. By limiting the number of connectable means that needs to be in fluid contact, the risk of contaminating the fluid path is reduced.

The infusion tubing 60 may, at its patient end opposite the reservoir 1, be connected to a catheter or an infusion set, e.g. of the kind disclosed in U.S. Pat. No. 5,968,011 (which is incorporated herein by reference). Alternatively, the infusion tube can be equipped with special closing means (not shown), e.g. in the form of a valve, or it may be simply closed by a clamp by providing a kink or a simple restriction of the tubing 60. Such clamps are well known in the art.

In the distal end 2 of the reservoir 1, sealing means 21 preferably formed in an annular ring 20 encircling the first or distal end 2 of the reservoir 1 is arranged. The annular ring 20 has a diameter greater than that of the body portion 4 of the reservoir. As can be appreciated from the figures in a preferred embodiment the reservoir is cylindrical, i.e. of circular cross section. However, the reservoir cross section could possibly have a number of different geometrical shapes. A rearward facing surface 21 of the annular ring 20 is adapted to sealingly cooperate with a rim 120 of the chamber 104 in the housing 101 of the infusion device 100. A gasket (not shown) e.g. formed in an elastomer may be provided between the surface 21 of the annular ring 20 and rim 120 of the chamber 104.

The sealing means 21 in combination with wall 5, and the closeable dispensing means 60 comprise a compact closure for chamber 104 of infusion device 100 preventing the entrance of dirt, moisture and water into the chamber 104 and the drive and control mechanism of the infusion device 100. This compactness of the distal end of the reservoir 1 provides for a large effective volume of the reservoir. Also the compactness of the distal end of the reservoir 1 provides for a relatively smooth front distal end 105 of the infusion device. This prevents the device from getting attached to the clothes of the patient and allows for easier placement of the device on the patient or on the patients clothes.

The reservoir 1 comprises connection means 40, 50 for securing the reservoir in a chamber 104 of the infusion device 100. The connection means may mean comprise a first set of connection means 40. Such means could advantageously comprise elevations 41 disposed on the side surface of the body portion 4 of the reservoir 1 corresponding with a threading (not shown) disposed in the chamber 104. Thus the reservoir 1 can be screwed into place in the chamber 104 of the infusion device 100. The threading might be of the interlocking type.

The first set of connection means 40 however could also be formed as a snap mechanism cooperating with means on the inside of infusion device 100 chamber 104. This could be preferably be utilized if a non circular cross section of the reservoir was chosen.

The reservoir 1 preferably additionally comprises a second set of connection means 50. Preferably, said second set of connection means 50 are disposed in connection with the sealing means 21. In a preferred embodiment the second set of connection means comprises at least one flexible arm 51 disposed on the annular ring 20. At the end of arm or arms 51 taps 52 are provided. Taps 52 cooperate with corresponding indentions (not shown) in the distal face 105 of the housing 101 of the infusion device 100. Thereby the reservoir is releasably locked or secured to the housing 101, because the reservoir is thus prevented from being rotated contrary to the threading. The inherent resiliency of arm(s) 51 secures that the reservoir can be released by rotating the reservoir against the threading direction by applying a suitable amount of force. A further advantage of the second set of connection mean 50 is that the connection means 50 can be applied to secure a desired rotational position of the reservoir, in case this is needed e.g. because of a special position of the drive member 102 for its connection to the piston 30 of the reservoir 1.

The reservoir 1 is adapted for containing and dispensing a fluid medical compound. This fluid medical compound may be filled through the dispensing means 60 and outlet 6, acting in this instance as an inlet. Alternatively the medical reservoir may be equipped with a separate inlet mechanism 70. This inlet mechanism 70 ay be comprised by an aperture 71 through wall 5 of the reservoir 1, closed by a piercable septum 72, preferably of a resilient material, e.g. an elastomer. The aperture 71 may be conducted through an elevation 73 on wall 5, said elevation 72 forming a stud, for allowing room for the septum 72. Thus the reservoir can be filled or refilled by pressing a hollow needle through the septum. When the needle is retracted the resiliency of the material of septum 72 will force the septum to close.

In a further embodiment of the invention the distal or first end 2 of the reservoir may be provided with a pressure vent 80. The pressure vent 80 is formed as an aperture in wall 5 opening against the distal side of wall 5 and the side of the distal end of body portion 4 of the reservoir. The aperture is provided with a membrane 82 for preventing dirt, etc to enter the chamber 104 of the infusion device, the membrane being permeable to air. The membrane may be formed in the material Gore Tex® or an equivalent thereto. The purpose of the pressure vent 80 is to allow for equalization of the atmospheric pressure outside the infusion device 100 and inside the chamber 104. A pressure difference might occur where a reservoir has been loaded at one atmospheric pressure say at sea level, where after the infusion is attempted used at a lower or higher pressure say in an airplane. Without a pressure vent, the pressure difference will apply a force on the piston, thus giving rise to inaccuracies in dosage.

It is therefore intended that the foregoing detailed description be regarded as illustrative rather than limiting, and that it be understood that it is the following claims, including all equivalents, that are intended to define the spirit and scope of this invention.

1. A medical reservoir (1) for a front loaded infusion device (100) comprising a housing (101), a chamber (104) inside said housing (101) for receiving said reservoir (1) and a drive member (102), said reservoir (1) comprising
a first end (2);
an outlet (6) disposed at said first end, said outlet having closable dispensing means;
a second open end (3);
an elongate body portion (4) disposed between said first and second ends (2, 3); and
a piston (30) slideably arranged within said elongate body portion (4) and being connectable to said drive member (102), wherein said reservoir (1) further comprises sealing means (21) disposed at the first end (2) of said reservoir (1) adapted for closing said chamber (104), at least one set of connecting means (40, 50) for securing the reservoir (1) in the housing (101).

2. A medical reservoir (1) according to claim 1 wherein said dispensing means is an infusion tube (60) fixedly arranged to the distal end (2) of the reservoir (1), and in fluid communication with said outlet (6).

3. A medical reservoir (1) according to claim 1 wherein said sealing means (21) comprise a rearwardly facing surface of an annular collar (20) disposed at the distal end (2) of the reservoir (1).

4. A medical reservoir (1) according to claim 1 wherein said connection means comprise a first set of connecting means (40), disposed on the outer surface of body portion (4).

5. A medical reservoir (1) according to claim 4 wherein said first set of connecting means (40) are disposed on the distal half of the outer surface of body portion (4).

6. A medical reservoir (1) according to claim 1 wherein said reservoir (1) comprises a second set of connecting means (50).

7. A medical reservoir (1) according to claim 6 wherein said second set of connecting means (50) are disposed on said collar (20).

8. A medical reservoir (1) according to claim 7 wherein said second set of connecting means (50) comprises at least one flexible arm (51) on said collar (20).

9. A medical reservoir (1) according to claim 1 wherein said reservoir further comprises a mechanism (70) for refilling the reservoir.

10. A medical reservoir (1) according to claim 1 wherein said reservoir further comprises a vent (80) for equalizing the pressure in a chamber of the infusion receiving said reservoir (1).

11. A medical reservoir (1) according to claim 10 wherein said vent (80) comprises a channel providing a connection between a front wall (5) and the side wall (4) of said reservoir (1).

12. A medical reservoir (1) according to claim 11 wherein said vent (80) further comprises membrane disposed at said front wall (5).
